(19) United States
(12) Patent Application Publication
(10) Pub. No.: US 2009/0163756 A1
(43) Pub. Date: Jun. 25, 2009

(54) Reactor cooler

(75) Inventor: Peter R. Pujado, Kildeer, IL (US)

Correspondence Address: HONEYWELL INTERNATIONAL INC;PATENT SERVICES, 101 COLUMBIA DRIVE, P O BOX 2245 MAIL STOP AB/2B, MORRISTOWN, NJ, 07962, US

(73) Assignee: UOP LLC, a corporation of the State of Delaware, Des Plaines, IL (US)

(21) Appl. No.: 12/002,814

(22) Filed: Dec. 19, 2007

(51) Int. Cl. C07C 1/00; B01J 19/00; F28D 15/00

(52) U.S. Cl. 585/638; 422/146; 165/104.28

(57) ABSTRACT

A reactor for converting a feedstream having one or more oxygenated compounds to a product containing olefins is provided. The reactor comprises a fluidized reaction zone defined by a reactor wall and a feedstream inlet located adjacent the reaction zone. The feedstream inlet is operative to feed the reaction zone with said feedstream. A riser extends from said reaction zone and carries a vaporized combination of said feedstream and said catalyst from said reaction zone to a disengaging zone fed by the riser. At least one cooling tube is disposed within the reactor and extends substantially vertically and substantially parallel to the reactor wall. The cooling tube is located adjacent the reactor wall and extends from an upper portion of the reaction zone towards a lower portion of the reaction zone. Also provided is a cooling system for a methanol to olefin reactor. Finally, a method of producing olefins from a feedstream having an oxygenate is provided. The method includes cooling the reaction which produces olefins from the oxygenate feedstream.

### TECHNICAL FIELD

This disclosure relates generally to processes and systems for use in cooling reactions within fluidized bed reactors. More particularly, the disclosure relates to hydrocarbon conversion processes utilizing a fluidized bed reaction zone and the reaction which results in the conversion of methanol to olefins.

### BACKGROUND

Processes and systems for converting feedstreams containing oxygenates to a product containing olefins are known. One example of such a process, using a fluidized bed reaction, is shown in U.S. Pat. No. 6,166,282. U.S. Pat. No. 6,166,282, which is incorporated herein by reference, shows an oxygenate conversion process and fast-fluidized bed reactor with an upper disengaging zone and a lower reaction zone. The reaction zone has a dense phase zone and a transition zone which extends upwardly from the dense phase zone into the disengaging zone. The feedstock, in the presence of an optional diluent is passed into the dense phase zone, which contains a non-zeolitic catalyst to effect at least a partial conversion of the feedstream to light olefins. The feedstream and catalyst are then passed into the transition zone to achieve essentially complete conversion. A portion of the catalyst is withdrawn from above the transition zone in the disengaging zone, at least partially regenerated through an external regenerator, and returned to a point above the dense phase zone, while catalyst is continuously circulated from the disengaging zone to the lower reaction zone. Additionally, the disengaged catalyst that is not regenerated is collected and recirculated into the dense phase zone via external recirculation standpipes. In this process, the temperature of the reaction is controlled by removing heat from the catalyst via external heat exchangers within the standpipes or adjacent the regenerators.

U.S. Pat. No. 4,071,573 teaches a process for prolonging the life of a catalyst by disposing of exothermic reaction heat. Owen discloses a vertical reactor vessel through which a feedstream and fluidizable catalyst particles are passed upwardly in a reaction. In one embodiment, the reaction temperature is controlled by applying a quench fluid to the reaction via distributor grids spaced vertically through the reactor. In another embodiment, the catalyst is cooled prior to injection into the reaction zone. In a final embodiment, there are a number of heat exchange tubes spaced vertically about the reactor. The heat exchange tubes form grids that span the reactor's cross section. Feedstream is passed through the heat exchange tubes, thereby removing heat from the reaction and preheating the feedstream. In addition, the catalyst used in the reaction is cooled prior to injection into the reactor.

In view of the need to control the temperature of reactions such as those described above, it could be beneficial to provide an efficient and cost effective way to cool such reactions.

### SUMMARY

I provide a reactor for converting a feedstream having an oxygenate to a product containing olefins. The reactor comprises a fluidized reaction zone defined by a reactor wall and a feedstream inlet located adjacent the reaction zone. The feedstream inlet is operative to feed the reaction zone with said feedstream. A riser extends from said reaction zone and carries a vaporized combination of said feedstream and said catalyst from said reaction zone to a disengaging zone fed by the riser. At least one cooling tube is disposed within the reactor and extends substantially vertically and substantially parallel to the reactor wall. The cooling tube is located adjacent the reactor wall and extends from an upper portion of the reaction zone towards a lower portion of the reaction zone. I also provide a cooling system for use in said reactor and a method of producing olefins using a reactor and cooling system.

### BRIEF DESCRIPTION OF THE DRAWINGS

FIG. 1 is a schematic diagram of a fluidized reactor.

FIG. 2 is a side elevational view of the interior wall of a reactor.

FIG. 3 is a top down cross sectional view of a reactor.

### DETAILED DESCRIPTION

My processes and systems are discussed in the context of using a representative reactor which employs a fast-fluidized bed reactor. Although the specifics of a fast-fluidized bed reactor are given for purposes of illustrating the significance of temperature control in a reactor, those skilled in the art will recognize the applicability of my processes and systems to reactions occurring in similar reactors. Below, an overview of a typical reactor and reaction process is given, along with detailed illustrations of a preferred a reactor employing a cooling system.

The illustrated fast-fluidized bed reactor comprises an upper disengaging zone and a lower reaction zone. The lower reaction zone comprises a dense phase zone which operates within a superficial velocity range less than about 1 meter per second (3 feet per second). By the term “superficial velocity”, it is meant the velocity of the gas as it flows through the vessel. The superficial velocity is typically determined by dividing the volumetric flow rate of the gas by the cross-sectional area of the vessel.

A transition phase zone is disposed above the dense phase zone and extends from the lower reaction zone into the upper disengaging zone. The transition phase zone includes a reducing cone which reduces the flow path diameter from the diameter of the dense phase zone to the diameter of the riser. The superficial velocity within the transition zone ranges between about 1 meter per second (3 feet per second) and about 6 meters per second (13 feet per second). A feedstream of feedstock at effective conditions is introduced into the lower reaction zone wherein it contacts a partially coked catalyst to selectively produce light olefins. As the unreacted feedstock and reaction products pass through the dense phase zone, they are carried into the transition zone with partially coked catalyst particles having a reduced number of active catalyst sites. As the mixture of unreacted feedstock, fluidized catalyst particles, and reaction products enters the transition zone, the reaction continues to essentially complete conversion (about 99 mole percent) of the oxygenate feedstock. At least one catalyst recirculation standpipe is provided to transfer or return a portion of the catalyst mixture from the upper catalyst bed to the dense phase zone.

Preferably, a catalyst cooling system is provided to cool the catalyst and feedstream as they react. The cooling system, as described in further detail below, comprises a plurality of cooling tubes disposed along the reactor wall within the reactor. The cooling tubes are fed with boiler feedwater which is heated to produce steam that is preferably used elsewhere in the reactor complex. The use of a cooling system employing cooling tubes within the reactor system eliminates the need for costly and complex flow through catalyst coolers.

Reaction conditions can be determined by those skilled in the art and preferably comprise a temperature of from about 200 degrees to 600 degrees Centigrade, more preferably from about 300 degrees to 500 degrees Centigrade, and a pressure of from about 1 to 200 psia, more preferably from about 20 to 100 psia. Typical processes for producing light olefins are described in U.S. Pat. Nos. 4,499,327 and 4,873,390 cited above and hereby incorporated by reference.

Preferably, the reaction zone comprises at least one catalyst recirculation standpipe to facilitate return of catalyst to the dense phase zone. A portion of the catalyst from the dense phase zone is withdrawn, optionally stripped in a conventional manner, and passed to a regeneration zone. In the regeneration zone, the coked catalyst is at least partially regenerated to produce a regenerated catalyst. The regenerated catalyst is returned to the reaction zone at a location above the dense phase zone. More particularly, the regenerated catalyst may be returned to the reaction zone at a location above the dense bed such as at a point in the riser or transition zone, or at a point in the disengaging zone such as to the upper catalyst bed. It is believed that by returning the regenerated catalyst to a location above the dense phase zone, contact between the freshly regenerated catalyst and the oxygenate feedstock is minimized, thereby improving selectivity to ethylene and the overall production of coke is reduced. The regenerated catalyst is lifted to the reaction zone with a portion of the net product stream. Preferably, the portion of the net product stream used to lift the regenerated catalyst comprises butene which was fractionated from the net product stream in a fractionation zone producing an ethylene stream, a propylene stream and a butylene stream.

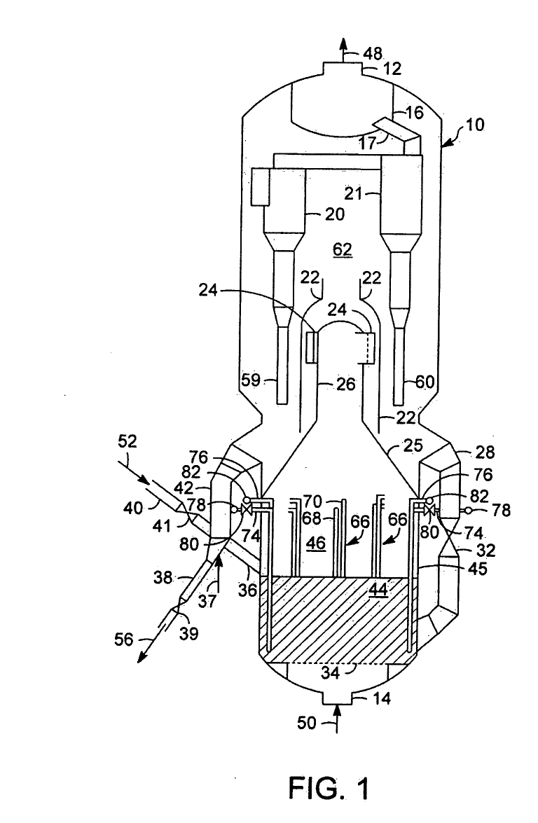

Referring now to FIG. 1, a fast-fluidized bed reactor 10 is illustrated in schematic form. The reactor 10 comprises a disengaging zone 62 and a lower reaction zone having a dense phase zone 44 and a transition phase zone 46, defined by a reactor wall 45. The reactor wall may be constructed of stainless steal, with a hardened liner for corrosion protection.

A feedstream is passed via line 50 to the feedstream inlet 14 in the presence of a diluent. The feedstream preferably comprises at least one oxygenate feedstock selected from the group consisting of methanol, ethanol, dimethyl ether, and the like. The feedstock and diluent admixture passes through a feed distributor 34 and enters the dense phase zone 44. The feed distributor 34 may consist of a uniformly flat sieve plate which permits the vapor phase feed admixture to pass through while retaining a catalyst above the sieve plate. Generally, the feed distributor 34 is supported by a ring having an overall diameter smaller than the outside diameter of the generally circular feed distributor 34. The ring may be supported by a cylinder with perforations or vents extending therethrough to prevent accumulation of catalyst against its side. The cylinder may be typically welded to the ring at right angles to the sieve plate to form a feed distributor assembly and the feed distributor assembly is rigidly disposed on the base of the lower reaction zone above the feed inlet 14. The ring serves to support the catalyst bed and to reduce vibrations in the feed distributor 34. Alternatively, a spider type distributor may be used.

The catalyst in the dense phase zone 44 and the transition phase zone 46 may comprise a non-zeolitic small pore catalyst such as SAPO-34, SAPO-17, and mixtures thereof. As the feedstock enters the dense phase zone 44, the feedstock contacts the non-zeolitic small pore catalyst and reacts at effective conditions to produce a reaction product stream. The reaction product stream typically comprise light olefins, including ethylene, propylene, and butylene. In the course of the reaction, a carbonaceous deposit is produced on the catalyst, reducing the activity of the catalyst. The reaction product stream and a catalyst mixture comprising active catalyst and some deactivated catalyst are conveyed into the transition phase zone 46 in an intermediate portion of the reaction zone.

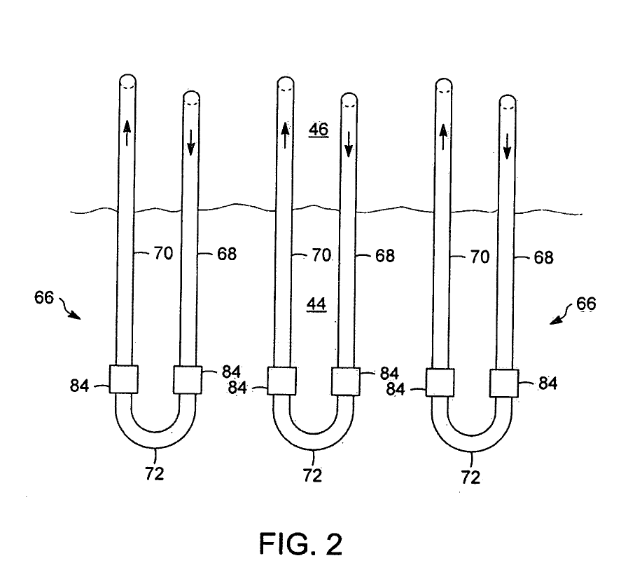

The reaction of the feedstock with the catalyst is exothermic, producing excess heat in the reactor 10. As with many reactions, it is important to keep the reactor 10 at a controlled, generally uniform temperature throughout the reaction process. Referring now to FIGS. 1 and 2, removal from the reactor 10 is facilitated by a cooling system 64. The cooling system 64 shown here comprises a plurality of cooling tubes 66 located within the reactor 10. The cooling tubes 66 have a generally “U” shaped configuration, with first tubes 68 and second tubes 70 connected to each other by a generally semicircular bottom portion 72.

Upper portions of the first and second tubes 68, 70 are fluidly connected to inlet tubes 74 and outlet tubes 76, respectively. The inlet and outlet tubes 74, 76 respectively supply and evacuate cooling medium to and from the cooling tubes 66. The inlet tubes are fed by a feedwater manifold 78, which supplies cooing medium to the first tube 68 via the inlet tube 74. Cooling medium passes through the first tube 68, the bottom portion 72 and the second tube 70. Upon exiting the second tube 70, the cooling medium flows through the outlet tube 76, which fluidly connects the second tube with an outlet manifold 82. Flow through the cooling tubes 66 may be controlled by a valve 80, which is positioned on the inlet tube 74. Locating valves 80 on inlet or outlet tubes 74, 76 allow flow control for individual cooling tubes 66. The valve 80 may be positioned anywhere along the flow path of the cooling medium to control flow of the cooling medium. By way of an alternative example, location of at least one valve 80 upstream from the individual tubes, such as along the feed manifold 78 allows an operator to control flow through the cooling tubes 66 with a single valve.

The feedwater manifold 78 and outlet manifold 82 may be segmented (e.g., in quadrants) so that, in case of a perforation or a leak, the faulty section can be isolated. Mixing in the fluidized bed is usually so vigorous that the temperature distribution remains uniform provided that the remaining cooling tube sections are sized to remove the excess heat.

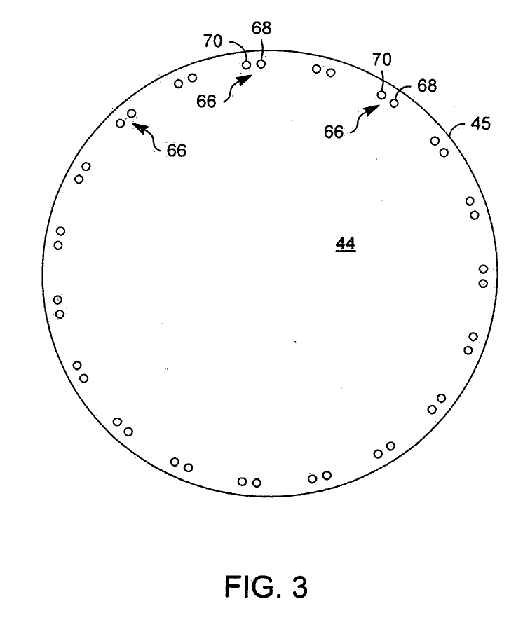

Referring now to FIG. 3, The cooling tubes 66 are vertically positioned along the reactor wall 45 and, in the structure shown, spaced evenly around the perimeter of the reactor 10. Because of the mixing characteristics of the catalyst and feedstream within the fluidized bed reactor, the perimeter location of the cooling tubes 66 facilitates even heat distribution throughout the reactor 10. Heat is transferred as the catalyst and feedstream mixture comes into proximity with the cooling tubes and is then transferred to the cooling medium.

As shown in FIGS. 1 and 2, the cooling tubes are positioned within the reactor 10 so that the lower portion of each cooling tube 66 is disposed within the dense phase zone of the reactor 10. Above the dense phase zone 44, the upper portion of the cooling tubes 66 also extend through the transition phase zone 46. The tubes 66 may also have mounts 84 which secure the tubes to the reactor wall 45 or to each other (not shown). The mounts 84 serve to hold the cooling tubes 66 in place and reduce vibration of the cooling tubes 66 within the reactor 10.

The cooling tubes 66 are preferably constructed of hardened steel to reduce the risk of corrosion which may occur within the reactor. Alternatively, the cooling tubes 66 may be constructed of any other material having the properties to withstand conditions occurring within the reactor 10. Although the structure shows cooling tubes 66 having a smooth surface, it may desirable to construct cooling tubes having a ribbed or finned surface or having a series of ridges located on the outer surface of the tube. Such surface features may serve to increase the surface area of the outer surface, thereby increasing heat transfer.

Heat is typically removed from the reactor 10, via the cooling tubes 66, to produce steam which can be used elsewhere in the complex. By way of example, steam produced within the cooling tubes may be used as a scrubber in connection with regenerating the catalyst.

As the reaction proceeds, the activity of the catalyst in the reaction zone gradually is reduced by the buildup of coke on the catalyst. As the reaction product and the catalyst mixture continue moving upwardly through the lower reaction zone into a riser section 26, the cross-sectional area of the flow path through the fast-fluidized bed reactor is reduced from the cross-sectional area of the dense phase zone 44 by a reducing means 25, or cone section, to the cross-sectional area of the riser section 26. In the fast-fluidized bed reaction system, the superficial velocity through the transition phase zone 46 varies between about 5 cm/s and 1 meter per second. The riser section 26 has a smaller diameter and a smaller cross-sectional area than the dense phase zone 44 which increases the superficial velocity through the riser relative to the dense phase zone 44. Because the superficial velocities in the riser section 26 are higher for the same feed rate, the cross-sectional area of the overall reactor zone can be decreased by about a factor of 2 to 3 times compared to the cross-sectional area of a bubbling bed reactor. In addition, the fast-fluidized bed reaction zone provides more precise control of the feedstock and catalyst rates without the need for external catalyst addition or removal. As a result, the fast-fluidized bed reaction system provides significantly decreased catalyst inventories over a bubbling bed reactor.

The reaction product stream and catalyst mixture continue to be conveyed through the riser section. The riser section discharges the reaction product stream and catalyst mixture through a separation zone consisting of distributor arms 24, or discharge opening, and a separation vessel 22. The discharge opening 24 tangentially discharges the reaction product stream and catalyst mixture to create a centrifugal acceleration of the catalyst and gas within the separation vessel 22 that provides an initial stage cyclonic separation. The catalyst mixture falls to the bottom of the disengaging zone 62 which defines a particle outlet for discharging fluidized catalyst particles and the vapor portion of the reaction product stream passes upwardly through a gas recovery outlet for withdrawing gaseous fluids from the separation vessel 22. Other configurations of separation zones may be suitable. The vapor, comprising entrained catalyst, continues upwards to a dilute phase separator typically in the form of a series of one to three conventional cyclone separation stages shown in the drawing as 20 and 21. Cyclone separation stage 20 represents a primary cyclone separation wherein a primary cyclone vapor stream is passed to a secondary cyclone separation stage 21 and the secondary vapors from the secondary cyclone separation stage 21 are conveyed via conduit 17 to a plenum chamber 16. Optionally, external cyclones bay be provided in addition to the cyclones shown within the reactor 10.

A net reaction product stream comprising less than about 100 ppm-wt catalyst is withdrawn via line 48 from the reactor outlet 12. Preferably, the net reaction product stream withdrawn from the fast-fluidized bed reaction zone comprises less than about 70 ppm-wt catalyst. Catalyst separated in the primary cyclone separation stage 20 drops through dip leg 59 into the bottom of the disengaging zone 62. Catalyst separated from the reaction product in the secondary cyclone separation stage falls through dip leg 60 into the bottom of the disengaging zone 62. Dip legs 59 and 60 are optionally fitted with flapper valves (not shown) at their base to prevent the back flow of vapors through the cyclone separators. Catalyst accumulated in the bottom of the disengaging zone 62 is allowed to achieve an upper catalyst level and any excess catalyst is passed through at least one external catalyst recirculation standpipe 28 through a recirculation slide valve 32, and returned to the dense phase zone 44. Preferably, at least two external catalyst recirculation standpipes are employed to return catalyst from the disengaging zone 62 to the dense phase zone 44.

To maintain the conversion and selectivity of the reaction at acceptable levels, a portion of the catalyst mixture is withdrawn as a spent catalyst stream from the upper disengaging zone 62 and passed through a spent catalyst standpipe 42. In the spent catalyst standpipe 42, the spent catalyst stream may be stripped with a stripping medium such as steam produced in the cooling tubes 66, which is introduced in line 37 to produce a stripped catalyst stream 56. The spent catalyst standpipe 42 typically includes a stripping section that contains grids or baffles to improve contact between the catalyst and the stripping medium. The stripped catalyst stream is conveyed through line 38 and the spent catalyst slide valve 39. The stripped catalyst stream 56 is passed to a catalyst regeneration zone (not shown).

In the catalyst regeneration zone, the spent catalyst stream is at least partially regenerated by oxidation to produce a regenerated catalyst stream. Such regeneration is well known to those skilled in the art of fluidized bed reaction systems. A regenerated catalyst stream 52 is returned to the lower reaction zone via a regenerated catalyst standpipe comprising line 40, regenerated catalyst slide valve 41, and line 36 to a point above the dense phase zone 44. The regenerated catalyst return is shown at a point above the dense phase zone. However, the return of the regenerated catalyst to the reaction zone may be provided at any point in the riser or in the upper catalyst bed. Preferably, the dense phase zone is operated to maintain a bed height of between about 2 meters (7 feet) and about 6 meters (20 feet) above the feed distributor 34 and below the intermediate portion of the reaction zone in the dense phase zone. More preferably, the bed height of the dense phase zone comprises between about 2.4 meters (8 feet) and about 4 meters (13 feet). By maintaining this bed height in the dense phase zone 44, it is believed that feedstock flow variations and “jet penetration” at the feed distributor are minimized to provide a well-mixed reaction zone comprising catalyst having a carbon content of between about 3 and 20 weight percent. It is believed that returning the regenerated catalyst to the point above the dense phase zone 44 improves the selectivity of the overall reaction toward ethylene and propylene. Freshly regenerated catalyst has the potential to crack the oxygenate feedstock to produce unwanted by-products. By contacting the feedstock with a partially regenerated catalyst in the dense phase zone and contacting the reaction products and unreacted material in the transition zone with a catalyst mixture which is a relatively more active catalyst mixture, the combination of spent catalyst with freshly regenerated catalyst, more complete conversion to the desired light olefin products is achieved.

The operating conditions depend, of course, on a particular conversion process and can be readily determined by those skilled in the art. Typical reaction parameters which control the reaction severity include temperature, space velocity, catalyst activity, and pressure. In general, reaction severity increases with increasing temperature, increasing catalyst activity, and decreasing space velocity. The effect of pressure on the reaction severity depends upon the particular reaction. Although any of the above described variables can be adjusted as necessary to obtain the desired hydrocarbon conversion, it is advantageous to have catalyst activity, directed to providing an effective amount of active catalyst sites within the moving bed reaction zone, to enhance the conversion to desired products while not enhancing the conversion to undesired by-products.

A variety of modifications to the structures and processes described will be apparent to those skilled in the art from the disclosure provided herein. Thus, the processes and systems may be in other specific forms without departing from the spirit or essential attributes thereof and, accordingly, reference should be made to the appended claims, rather than to the foregoing specification, as indicating the scope of my disclosure.

1. A reactor for converting a feedstream having one or more oxegenated compounds to a product containing olefins comprising:
a fluidized reaction zone defined by a reactor wall;
a feedstream inlet located adjacent the reaction zone and operative to feed the reaction zone with said feedstream;
a riser extending from said reaction zone that carries a vaporized combination of said feedstream and said catalyst from said reaction zone;
a disengaging zone fed by the riser; and
at least one cooling tube disposed within the reactor and extending substantially vertically and substantially parallel to the reactor wall, the cooling tube being located adjacent the reactor wall and extending from an upper portion of the reaction zone towards a lower portion of the reaction zone.

2. The reactor according to claim 1, further comprising a cooling inlet extending through the reactor wall, the cooling tube further comprising a first end portion fluidly connected to said cooling inlet.

3. The reactor according to claim 2, further comprising a cooling outlet extending through the reactor wall, the cooling tube further comprising a second end portion fluidly connected to said cooling outlet.

4. The reactor according to claim 3, wherein said cooling inlet and cooling outlet are located adjacent each other proximate the upper portion of the reaction zone and the cooling tube has a generally U-shape configuration and extends downwardly from said cooling inlet and cooling outlet towards a lower portion of said reaction zone.

5. The reactor according to claim 1, wherein the cooling tube is at least partially constructed from hardened steel.

6. The reactor according to claim 1, further comprising a plurality of cooling tubes located within the reaction zone.

7. The reactor according to claim 6, wherein substantially all cooling of said catalyst is done by the cooling tubes.

8. A cooling system for a methanol to olefin reactor, the reactor having a shell defining a fluidized bed for reaction comprising:
an inlet extending through the shell and having an interior inlet portion located within the reactor and an exterior inlet portion located outside of the reactor;
an outlet extending through the shell and having an interior outlet portion located within the reactor and an exterior outlet portion located outside of the reactor; and
at least one cooling tube having a first end portion fluidly connected to the interior inlet portion, a second end portion fluidly connected to the interior outlet portion, and a tubular body fluidly connecting the first and second end portions and extending substantially downwardly from said inlet and outlet, proximate said reactor shell.

9. The cooling system according to claim 8, further comprising a plurality of inlet tubes, a plurality of outlet tubes and a corresponding plurality of cooling tubes fluidly connecting each inlet tube to an outlet tube.

10. The cooling system according to claim 8, wherein the exterior inlet portion is fluidly connected to a source of boiler feed water and the exterior portion of said outlet is fluidly connected to a heat reservoir.

11. The cooling system according to claim 8, wherein the reactor facilitates a fluidized bed reaction having a dense phase zone and wherein, the inlet and outlet are located above the dense phase zone and the cooling tube has a first portion extending downwardly from the inlet into the dense phase zone and a second portion extending upwardly from the dense phase zone to the outlet.

12. The cooling system according to claim 11, wherein the first and second cooling tube portions are joined by a transition piece located within the dense phase zone.

13. The cooling system according to claim 11, further comprising a plurality of inlets, outlets and cooling tubes spaced around the reactor shell.

14. A method of producing olefins from a feedstream having an oxygenate comprising:
providing a fast fluidized bed reactor having
a reactor wall defining a reaction chamber having at least a dense phase zone and a transition phase zone,
a feedstream inlet,
a cooling inlet and a cooling outlet each extending through the reactor wall adjacent the transition phase zone, and
a cooling tube extending downwardly from the cooling inlet to a point within the dense phase zone and back to the cooling outlet;
introducing a feedstream containing oxygenates to the inlet for injection into the reaction chamber;
contacting the feedstream with a non-zeolitic catalyst in the reaction chamber; and
introducing a cooling medium into the cooling inlet, the cooling tube and the cooling outlet, whereby heat from the reaction of the feedstream with the catalyst is transferred to the cooling medium.

15. The method according to claim 13, wherein the cooling medium is boiler feedwater.

16. The method according to claim 14, wherein the boiler feedwater is converted to steam within the cooling tube and steam flows through the cooling outlet.

17. The method according to claim 15, further comprising contacting at least a portion of the steam produced in the cooling tube with at least a portion of the catalyst to at least partially regenerate the catalyst.

18. The method according to claim 14, wherein substantially all of the cooling in said method is done by the step of introducing a cooling medium into the cooling inlet, the cooling tube and the cooling outlet.
